(19) United States
(12) Patent Application Publication
(10) Pub. No.: US 2019/0175893 A1
(43) Pub. Date: Jun. 13, 2019

(54) METHOD OF DELIVERING THERAPEUTIC SUBSTANCE TO THE SKIN

(71) Applicant: Karyn Grossman, Santa Monica, CA (US)

(72) Inventor: Karyn Grossman, Santa Monica, CA (US)

(21) Appl. No.: 16/213,583

(22) Filed: Dec. 7, 2018

Related U.S. Application Data
(60) Provisional application No. 62/596,051, filed on Dec. 7, 2017.

(51) Int. Cl. A61M 37/00; A61B 18/20; A61B 18/14; A61K 31/765; A61K 35/19; A61K 31/573

(52) U.S. Cl. CPC A61M 37/0015; A61B 18/20; A61B 18/1477; A61B 2018/0047; A61K 35/19; A61K 31/573; A61K 31/765

(57) ABSTRACT

A method of providing dermatological treatment at an under-eye region of a skin of a patient is provided. The method includes creating a plurality of micro-channels in a surface of the skin of the patient at the under-eye region and applying a substance topically to the skin of the patient at the under-eye region. The substance includes a therapeutic agent, which is delivered to the skin of the patient through the plurality of micro-channels. The therapeutic agent may be selected from the group consisting of a filler, a bio-stimulant, cortisol, and a combination thereof.

### CROSS-REFERENCE TO RELATED APPLICATION(S)

This application claims priority to and the benefit of U.S. Provisional Application Ser. No. 62/596,051, filed Dec. 7, 2017, the entire contents of which are incorporated herein by reference.

### BACKGROUND

Healthcare providers perform dermatological procedures on patients for many reasons, cosmetic and therapeutic, including improving the health, volume, and appearance of skin. Some such procedures which involve delivery of a therapeutic substance to the skin of the patient require the healthcare provider to provide localized injections of the therapeutic substance at the treated area. In addition, certain areas of a patient's skin may be particularly sensitive and/or may respond differently to different dermatological procedures. There is an ongoing need to identify treatments which provide or enhance the desired improvements in the health, volume, and appearance of the skin, and/or which are particularly effective at specified locations on the patient's body.

### SUMMARY

Aspects of the present disclosure are directed to a method of treating the skin of a patient. A procedure is performed on the skin of the patient, creating micro-channels in the skin. A drug is applied topically to the channels in the skin, delivering the drug into the treated skin.

Aspects of the present disclosure are directed to a method of providing dermatological treatment at an under-eye region of a skin of a patient. The method includes creating a plurality of micro-channels in a surface of the skin of the patient at the under-eye region, and applying a substance topically to the skin of the patient at the under-eye region, the substance containing a therapeutic agent, the therapeutic agent being delivered to the skin of the patient through the plurality of micro-channels, wherein the therapeutic agent is selected from the group consisting of a filler, a bio-stimulant, cortisol, and a combination thereof.

In some embodiments, the therapeutic agent is a collagen stimulator.

In some embodiments, the therapeutic agent is configured to induce collagen synthesis in the skin in the under-eye region.

In some embodiments, the therapeutic agent is poly-l-lactic acid.

In some embodiments, the therapeutic agent is applied in an amount cosmetically effective to increase the volume of the skin in the under-eye region.

In some embodiments, the therapeutic agent is platelet rich plasma.

In some embodiments, the therapeutic agent is configured to stimulate hyaluronic acid.

In some embodiments, creating the plurality of micro-channels is performing a fractional treatment having a treatment density between 5-40%.

In some embodiments, creating the plurality of micro-channels is performing a needle-based or RF needle based fractional treatment.

In some embodiments, creating the plurality of micro-channels is performing a laser fractional treatment.

These and other features and aspects of the present invention will be more fully understood when considered with respect to the following detailed description, appended claims, and accompanying drawings.

### BRIEF DESCRIPTION OF THE DRAWINGS

The accompanying drawings, together with the specification, illustrate preferred and example embodiments of the present invention, and, together with the description, serve to explain the principles of the present invention.

FIG. 1 is a flowchart showing a method of delivering a therapeutic substance to the skin according to embodiments of the present disclosure.

FIG. 2 is a treatment of an under-eye region according to embodiments of the present disclosure.

### DETAILED DESCRIPTION

Hereinafter, example embodiments will be described in detail. The present invention, however, may be embodied in various different forms, and should not be construed as being limited to only the illustrated embodiments herein. Rather, these embodiments are provided as examples so that this disclosure will be thorough and complete, and will fully convey the aspects and features of the present invention to those skilled in the art. Accordingly, processes, elements, and techniques that are not necessary to those having ordinary skill in the art for a complete understanding of the aspects and features of the present invention may not be described.

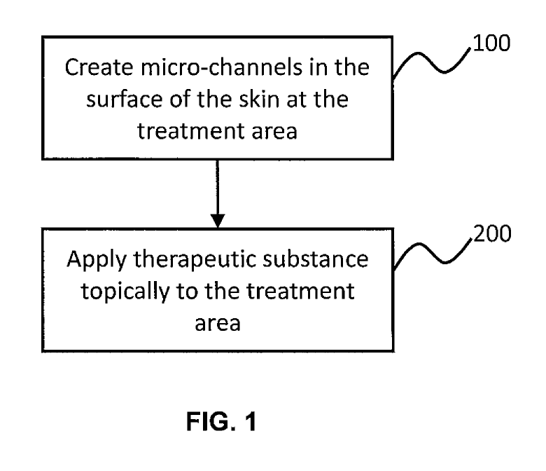

FIG. 1 is a flowchart depicting a method of delivering a therapeutic substance to the skin according to embodiments of the present disclosure. In act 100, micro-channels are created in the surface of the skin at the treatment area. In treatment methods according to embodiments of the present disclosure, a procedure is performed on a treatment area of a patient's skin. The procedure may be any procedure which causes channels and/or micro-channels (hereinafter ‘micro-channels’) in the treated portion of the skin, such as a factional wound. For example, an energy based device or a mechanical device may be used to create disruptions (e.g., small holes, microthermal zones, or areas of damaged cells) of the epidermis and/or dermis. In some embodiments, the micro-channels may not extend beyond the dermis. In some embodiments, the micro-channels may span from the epidermis all the way into the fat. A clinician performing the procedure may select a predetermined depth for the micro-channels, and the procedure may create micro-channels having the predetermined depth. The micro-channels may be numerous and in close proximity to each other, and distributed across the entire area of the treatment area. In some embodiments, the procedure may be a fractional laser treatment or a micro-needling treatment, including a fractional resurfacing treatment such as Fraxel, Active FX, Deep FX, Max FX, or Total FX or a needled radio frequency treatment such as Fractora. In some embodiments, the procedure may be a fractional treatment having a treatment density between 20% and 80%. In some other embodiments, the procedure may be a fractional treatment having a treatment density between 30% and 70%. In some other embodiments, the procedure may be a fractional treatment having a treatment density between 40% and 60%. In some embodiments, the procedure may be a fractional treatment having a treatment density between 5% and 10%.

In some embodiments, the procedure is performed on a treatment area characterized by thin skin. For example, in some embodiments, the treatment area is an under-eye region. In other embodiments, the treatment area may be an area of the face, neck, chest, torso, or extremities. In some embodiments, the procedure is performed on a treatment area characterized by scar tissue (e.g. atrophic scars or acne scars) or striae (e.g. stretch marks).

In act 200, a therapeutic substance is applied topically to the treatment area. The substance may contain a therapeutic agent. The therapeutic agent may be delivered to the skin of the patient through the plurality of micro-channels. The substance and/or agent may be a liquid, suspension, gel, lotion, or solid. In some embodiments, the substance is applied within 12 hours of performing the procedure causing the micro-channels. In some embodiments, the substance is applied immediately after the procedure is performed. The micro-channels created in the skin at the treatment area act as a conduit for the substance, allowing the substance to travel from the surface of the skin down the micro-channels, permeating the skin at the treatment area. The micro-channels allow the substance to penetrate further into the skin than might otherwise be possible through diffusion alone. The predetermined depth of the micro-channels allows the substance to penetrate to a predetermined depth. The close proximity of the micro-channels to each other and their distribution throughout the treatment area allows the substance to disperse substantially uniformly throughout the treatment area.

In some embodiments, the substance includes a filler substance. The filler may increase the volume of the skin. The filler may mimic or be a constituent of parts of the skin or body, or may provide a scaffolding of non-living materials to the skin or subcutaneous tissues but may not be innately found within the body. For example, the filler may include poly-L-lactic acid (e.g. Sculptra). The micro-channels allow the filler to disperse into the skin at a chosen concentration and depth, which can allow a more diffuse response (or diffused response) than seen in localized injections. The filler substance is delivered into the skin in a more uniform pattern (e.g. both parallel and perpendicular to the surface of the skin), which can obtain different results than would be obtained from direct injection into or below the skin. The channels may allow the substance to penetrate deeper into the skin along their channels than would be obtained from topical application alone, which can also obtain different results.

In some embodiments, the substance includes a bio-stimulant. The bio-stimulant may stimulate the body or skin to produce components of the body or skin, and may alter the cells' production or metabolism of enzymes or proteins, or alter other functions of the cells. For example, the bio-stimulant may induce collagen synthesis in the skin. The bio-stimulant may influence or affect the skin, including the epidermis, dermis, or subdermis or may act on organs, glands or structures within or below the skin. The bio-stimulant may affect distant sites. In some embodiments, the bio-stimulant may be platelet rich plasma derived from the patient's own blood. As discussed above with respect to filler substances, the application of a bio-stimulant according to embodiments of the present disclosure may allow the bio-stimulant to penetrate deeper than would be possible through diffusion alone, to a controlled depth, at a controlled concentration, and may allow the bio-stimulant to be distributed uniformly (or substantially uniformly) across the treatment area and along the depth of the skin.

In some embodiments, the substance contains a combination of a filler and a bio-stimulant (e.g., Scupltra), and may dissolve then encourage collagen to be formed. In one embodiment, the substance includes a combination of poly-L-lactic acid and platelet rich plasma. In some embodiments, the substance includes a constituent of the skin, such as hyaluronic acid.

In some embodiments, the substance includes cortisone. In one embodiment, the treatment area includes or is substantially composed of scar tissue. A fractionated procedure is performed on the scar tissue such that micro-channels are generated in the scar tissue. The depth of the micro-channels may be controlled such that the micro-channels do not extend beyond the scar tissue. The micro-channels may be created in the scar tissue but not in adjacent tissue according to an embodiment. A substance including cortisone may be applied topically to the scar tissue. The micro-channels may thereby distribute the cortisone throughout the scar tissue without allowing distribution into adjacent unscarred tissue. Conventional methods of applying cortisone to the skin via injection may present a risk of atrophy or thinning of the skin, for example as a result of injections that are too deep or from boluses into the skin. Application of cortisone according to embodiments of the present disclosure allows for a controlled and even penetration and distribution of the cortisone which may improve the response to the treatment and reduce the risk of atrophy seen in conventional methods.

In some embodiments, the substance includes one or more vitamins and/or one or more antioxidant, such as ferulic acid (e.g. a ferulic acid serum such as C E Ferulic).

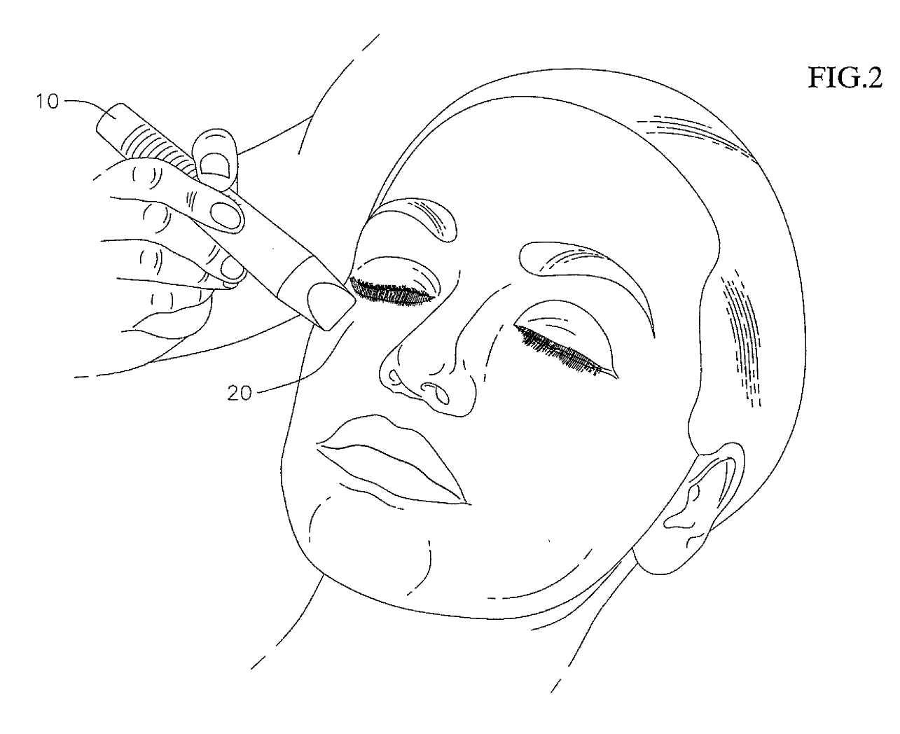

FIG. 2 shows a treatment of an under-eye region according to embodiments of the present disclosure. A clinician uses fractional device 10 to perform a fractional procedure to create a fractional wound in the under-eye region 20. In some embodiments, the under-eye region may be the region of the skin of the patient directly under the eye which is thinner than adjacent areas. The density of the fractional procedure and/or fractional wound may be between 5% and 40%.

Upon completing the fractional treatment (e.g., immediately following or substantially immediately following completing the fractional treatment), the clinician applies a therapeutic substance, such as those described above, to the fractional wound in the under-eye region 20. In particular, in some embodiments, the therapeutic substance applied to the under-eye region 20 is poly-l-lactic acid, a collagen stimulant, and/or a hyaluronic acid stimulant.

The terminology used herein is for the purpose of describing particular embodiments and is not intended to be limiting of the inventive concept. As used herein, the singular forms “a” and “an” are intended to include the plural forms as well, unless the context clearly indicates otherwise. It will be further understood that the terms “comprises,” “comprising,” “includes,” and “including,” when used in this specification, specify the presence of the stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list.

As used herein, the term “substantially,” “about,” and similar terms are used as terms of approximation and not as terms of degree, and are intended to account for the inherent variations in measured or calculated values that would be recognized by those of ordinary skill in the art. Further, the use of “may” when describing embodiments of the inventive concept refers to “one or more embodiments of the inventive concept.” As used herein, the terms “use,” “using,” and “used” may be considered synonymous with the terms “utilize,” “utilizing,” and “utilized,” respectively. Also, the term “exemplary” is intended to refer to an example or illustration.

Unless otherwise defined, all terms (including technical and scientific terms) used herein have the same meaning as commonly understood by one of ordinary skill in the art to which the present inventive concept belongs. It will be further understood that terms, such as those defined in commonly used dictionaries, should be interpreted as having a meaning that is consistent with their meaning in the context of the relevant art and/or the present specification, and should not be interpreted in an idealized or overly formal sense, unless expressly so defined herein.

While this invention has been described in detail with particular references to illustrative embodiments thereof, the embodiments described herein are not intended to be exhaustive or to limit the scope of the invention to the exact forms disclosed. Persons skilled in the art and technology to which this invention pertains will appreciate that alterations and changes in the described structures and methods of assembly and operation can be practiced without meaningfully departing from the principles, spirit, and scope of this invention, as set forth in the following claims and equivalents thereof.

1. A method of providing dermatological treatment at an under-eye region of a skin of a patient, the method comprising:
creating a plurality of micro-channels in a surface of the skin of the patient at the under-eye region; and
applying a substance topically to the skin of the patient at the under-eye region, the substance containing a therapeutic agent, the therapeutic agent being delivered to the skin of the patient through the plurality of micro-channels,
wherein the therapeutic agent is selected from the group consisting of a filler, a bio-stimulant, cortisol, and a combination thereof.

2. The method of claim 1, wherein the therapeutic agent is a collagen stimulator.

3. The method of claim 1, wherein the therapeutic agent is configured to induce collagen synthesis in the skin in the under-eye region.

4. The method of claim 1, wherein the therapeutic agent is poly-l-lactic acid.

5. The method of claim 4, wherein the therapeutic agent is applied in an amount cosmetically effective to increase the volume of the skin in the under-eye region.

6. The method of claim 1, wherein the therapeutic agent is platelet rich plasma.

7. The method of claim 1, wherein the therapeutic agent is configured to stimulate hyaluronic acid.

8. The method of claim 1, wherein creating the plurality of micro-channels is performing a fractional treatment having a treatment density between 5-40%.

9. The method of claim 1, wherein creating the plurality of micro-channels is performing a needle-based or RF needle based fractional treatment.

10. The method of claim 1, wherein creating the plurality of micro-channels is performing a laser fractional treatment.
